(19) United States
(12) Patent Application Publication
(10) Pub. No.: US 2005/0261646 A1
(43) Pub. Date: Nov. 24, 2005

(54) Ostomy appliance

(76) Inventors: Wayne Conrad, Hampton (CA); Brock Masters, Hampton (CA)

Correspondence Address: BERESKIN AND PARR, 40 KING STREET WEST, BOX 401, TORONTO, ON, M5H 3Y2, CA

(21) Appl. No.: 10/847,621

(22) Filed: May 18, 2004

(51) Int. Cl. A61F 5/44

(52) U.S. Cl. 604/338

(57) ABSTRACT

An ostomy bag in which an ostomy bag is supported on the user's skin by a combination of an inner sealing means to secure the stoma in the opening of the ostomy bag and an outer securing member spaced from the inner sealing means. The outer securing member is positioned adjacent the outer perimeter of the bag to secure the bag to the skin of a user. A water transport and or a chafing protection layer may be provided between the skin of the user and the ostomy bag.

### FIELD OF THE INVENTION

The present invention relates to ostomy appliances and in particular to the portion of the ostomy appliance used to attach the appliance to the body of the user.

### BACKGROUND OF THE INVENTION

A person whose normal bowel function or bladder is impaired due to a birth defect, injury, disease, or other disorder may undergo surgery known as an ostomy. Ostomy surgery often involves removal or bypass of impaired or diseased tissue and the creation of a hole in the abdominal wall of the patient, and pulling a section of the bladder conduit or bowel to the skin surface and subsequently attaching it to the skin surface. Bowel attachment surgery is termed an ileostomy when the small intestine is involved, termed a colostomy when the large intestine is involved, and termed a urostomy when the bladder is involved.

The hole in the skin to which the end of the bladder conduit or intestine is attached is called the “stoma,” and the surrounding skin area is called the “peristomal area”. The ileostomy and colostomy stoma provides an open conduit through which a constant or intermittent efflux of fecal material can occur and the urostomy stoma allows the efflux of urine. The urine or feces is collected in a bag-like ostomy appliance, which is worn by the user and replaced by the user as required every few days. In this application, the word ostomy is used to refer to an ileostomy, colostomy, urostomy or any like procedures, which now exist or which may be developed in the future and which result in the efflux of urine and/or fecal matter through a surgically created opening in the skin of a person.

The attachment of the ostomy appliance to the user can be achieved with either a one piece or a two-piece system. A two-piece system typically consists of a flange, which is adhered to the body of the person who has an ostomy, and a bag like device to provide collection of the feces or urine. A mechanical member allows the bag to be removably fastened to the flange and for a plurality of bags to be used with a single flange over a period of 2-7 days. The one-piece system integrates into one-piece construction both the means of adhering to the body and the bag collection device.

Current adhesive systems for ostomy appliances which are in use typically consist of an inner hydrocolloid layer over which a protective paper or cloth tape is adhered to provide a barrier through which feces and urine will not pass but which does allow oxygen and water vapor to pass. The barrier must have a hole cut in it to match the stoma of the person wearing it as stomas of different people vary in size and shape.

Current adhesive systems for ostomy appliances often become detached from the body of the user if the user sweats or if the product is exposed to moisture, water or excessive heat. Many bags incorporate a means of attaching a belt which users may optionally employ to help keep the bag from pulling off or to support a hernia. However, ostomy appliances still become detached if sufficient water is present. Accordingly, a problem with current ostomy appliances is that sweat, moisture or heat or a combination thereof compromises the integrity of the seal against the body, resulting in users being concerned about the embarrassment and inconvenience of the ostomy appliance leaking or becoming detached. A further problem is that the bag may become dislodged due to a force applied to the bag during intense exercise or contact sports.

### SUMMARY OF THE INVENTION

As is known in the art, an adhesive member or layer is provided in an annular band around the entrance for the stoma. This band or layer is utilized to secure the bag to a person such that the stoma is in fluid flow communication with the interior of the ostomy bag. As the ostomy bag fills with fluid or fecal matter, the weight of the contents of the bag will pull the bag downwardly (if the person is in an erect position). This applies strain to the adhesive band or layer around the entrance for the stoma. In accordance with one aspect of the instant invention, an ostomy bag is provided with an outer securing member that removably affixes the ostomy bag to the skin of the user at a position distal to the entrance for the stoma. The outer securing member supplements the support provided by the adhesive band or layer around the entrance for the stoma. The securing member is spaced from the adhesive system used with current one or two-piece bags and, accordingly, is applicable to both one-piece and two-piece systems.

It will be appreciated that the securing member may be provided inwards of the outer perimeter of the bag and outwardly of the adhesive band or layer around the entrance for the stoma. The closer the securing member is positioned to the outer perimeter of the bag, the thinner the band may be, or the weaker the adhesive, to support the same weight in the bag. An advantage of the present invention is that the additional support of the ostomy bag offers protection from mechanical detachment of the bag during work, sports or intimacy.

While a user is wearing a bag, an object may become inserted between the bag and the skin of the user causing the bag to be pulled off. This may occur, for example, during sports or intimacy when another person's hand may be placed between the bag and the user. Accordingly, the securing member is preferably positioned at the outer perimeter of the bag so as to secure the outer perimeter of the bag to the user and prevent a foreign object being positioned between the bag and the user. More preferably, the outer securing member is on the outer face of the ostomy bag and extends outwardly to overlie the outer perimeter of the ostomy bag.

In one embodiment, the securing member may be provided adjacent only one side of the perimeter of the bag. In another embodiment, the securing member may be positioned generally adjacent a substantial portion of the outer perimeter of the bag and, preferably, essentially adjacent the entire outer perimeter of the bag. The securing member may be continuous or discontinuous.

When an ostomy bag is placed flush against the skin of a user, sweat will accumulate behind the bag. This arises since, by design, the bag is impermeable to fluids. Accordingly, when the bag is adjacent the skin of a user, the impermeability of the bag causes sweat to pool on the skin against which the bag sits flush. In accordance with another aspect of the instant invention, a water transport layer is placed between the surface of the ostomy bag that faces a user and the skin of the user. The water transport layer permits water to be transported in a plane generally parallel to the skin of the user towards, and preferably to, the outer perimeter of the bag. At the outer perimeter, the sweat may evaporate. The water transport layer may be a woven or non-woven material, which permits water to be transported through the material. Another advantage of the use of a water transport layer is that it also permits gases such as oxygen and carbon dioxide to flow therethrough so that the skin can breathe.

In accordance with another advantage of the instant invention, an abrasion or chafing protection layer may be placed between the surface of the ostomy bag that faces a user and the skin of the user. This layer may be a soft material that prevents the bag from damaging the skin of the user when the bag rubs against the skin of the user. This layer may be provided only adjacent the peristomal area or it may be provided along all, or essentially all, of the portion of the surface of the bag that faces the user outwards of the adhesive member or layer that is provided around the entrance for the stoma. The layer may be made of a woven cloth.

In one preferred embodiment the abrasion or chafing protection layer also functions as a water transport layer.

The water transport layer and/or the chafing protection layer may be made from natural fibers, synthetic fibers or a blend of natural and synthetic fibers. Preferably, the layer is a woven or non-woven cotton or cotton polyester blend. However, it will be appreciated that fabrics that are able to wick water away from the skin have been used with clothing for running and any such materials which are known in the art may be used.

The outer securing member may be made of a material that is permeable to water vapor and gasses such as oxygen and carbon dioxide and/or the outer securing member may be discontinuous so as to leave openings through the outer securing member. Thus, even with the outer securing member in place, sweat may be permitted to leave the area between the ostomy bag and the skin in that region may breathe. For example. If the outer securing member is permeable to gasses, then oxygen may permeate through the securing member. Alternately, if the outer securing member is not permeable to gasses, then the outer securing member may be applied discontinuously so as to provide openings through the outer securing member.

In another embodiment, the outer securing member is impermeable to liquid water. Such an application may be utilized if the user is going swimming. In such a case, the outer securing member is preferably permeable to gasses and water vapour. For example, the outer securing member may be made from urethane. In such an embodiment, the use of an abrasion or chafing protection layer is advantageous to protect the skin behind the ostomy bag during exercise. Further, if the outer securing member is permeable to water vapour, a water transport layer may also be used.

In the application of either the one piece or the two-piece ostomy appliance, the currently used hydrocolloid layer and adhesives guard against infection and skin irritation and affix the bag to the skin adjacent the stoma. The outer securing layer supports the ostomy bag to enhance the likelihood that the hydrocolloid will remain in place and not be adversely impacted or degraded by body fluids or water.

The outer securing member, which may be for example, a urethane tape layer, may provide a thin flexible oxygen and water vapor permeable but liquid water and body fluids impermeable layer while providing a supplemental attachment between the body and the ostomy bag of either one and two piece systems. The optional water transport and/or chafing protection layer, which may be paper or cloth tape layers, that are placed inside the waterproof perimeter seal of the urethane tape are also protected from degradation.

The outer securing member for the ostomy bag may employ a segmented or intermittent adhesive layer so that the outer securing layer is applied discontinuously around the outer perimeter of the ostomy bag and is therefore equally applicable to either one or two-piece bags.

The adhesive of the outer securing member may be any adhesive that is known in the bandage art. The adhesive is generally unaffected by water, i.e., it generally does not dissolve or soften or swell or lose its adhesion when exposed to water, and it is permeable to water vapor and to oxygen. There are a number of adhesives suited to such application including but not limited to acrylate adhesives, vinylacrylate adhesives and polyvinyl ether adhesives. The membrane-adhesive combination of this invention is preferably very moisture and oxygen permeable compared to paper and cloth tapes commonly used against the skin in current ostomy appliances. This permeability allows evaporation of perspiration from the skin beneath the membrane helping the skin to remain dry and healthy and helping to minimize bacterial growth on the skin. This permeability allows oxygen to reach the skin, an important factor in promoting healing of any lesions on the skin and in promoting the bacteria fighting mechanisms of the skin.

3M Tegaderm™ membrane, which is the preferred urethane membrane material of this invention, is impermeable to bacteria and to viruses such as HIV. In a preferred embodiment of this invention the membrane is a continuous material made of polyurethane elastomer which can be manufactured in a manner which provides the desired thinness, flexibility, stretchability, and permeability to water vapor and oxygen while being compatible for adhesion to and interaction with the ostomy bag and the skin of the user.

Adhesive coated membranes having these desirable properties of thinness, low modulus of elasticity, and permeability have been in use for over thirty years for burn and wound dressings and as attachments for some ostomy appliances. Dressings of this general type are sold under a number of trade names including “Op-Site” made by Smith & Nephew, Tegaderm™ made by 3M, Bioclusive™ made by Johnson & Johnson, Thinfilm™ made by Hollister and Uniflex™ made by Howmedica. This type of material is described in U.S. Pat. No. 3,645,835, assigned to Smith & Nephew, issued Feb. 29, 1972, which is incorporated herein by reference. Op-Site is sold by Smith & Nephew under this patent, and Tegaderm and Bioclusive are sold under the same patent by virtue of license agreement with Smith & Nephew.

In another embodiment, the membrane may be a non-continuous material including fabrics to which an adhesive suitable for use with skin is applied. Fabrics that may be used include, but are not limited to, non-woven fabrics based on cellulose or synthetic polymer fibers, which may be crimped and/or laid down in such a manner as to give an elastic fabric. One type of fabric having the desirable properties of omni-directional stretch combined with high porosity and high strength, even when wet, is that type of fabric based on extruded composite synthetic fibers sold under the trade mark Heterofil™ by Imperial Chemical Industries Limited. The skin of the abdomen can readily stretch by 30% although most of the occasions of stretching will be significantly less than that. It follows that an attachment system that adheres to the skin should also be able to be stretched by the attached skin by at least 30% in any direction, and do so without exerting undue tugging force on the skin which would be uncomfortable and tend to break the bond on the skin. The membranes used in this invention are preferably capable of stretching to elongate them substantially elastically by 20% when no more than 1.6 pounds/inch is applied to the membrane, and preferably less than 0.6 pounds/inch.

The ostomy appliances of the present invention may be useful for periods of up seven days for users engaged regularly in physical activity. In addition, ostomy appliances of the present invention may be used while a user is exercising, including swimming, martial arts and skiing, and also by a user when in a hot tub, steam room and sauna attendances.

In accordance with one embodiment of the instant invention, there is provided an ostomy appliance comprising an inner face having an opening which is adapted for receiving a stoma, an outer perimeter, an opposed outer face, an inner sealing member positioned adjacent the opening and an outer securing member spaced from the inner sealing member.

In one embodiment, the outer securing member is positioned adjacent the outer perimeter.

In another embodiment, the outer securing member is positioned on the outer face.

In another embodiment, the outer securing member extends from a position on the outer face inwards of the outer perimeter and extends outwardly to a position beyond the outer perimeter.

In another embodiment, the outer securing member is provided at discrete locations around the inner sealing member.

In another embodiment, the outer securing member extends in a continuous annular band around and spaced from the inner sealing member. The outer securing member may have openings provided therein.

In another embodiment, the outer securing member is impermeable to liquid water.

In another embodiment, the outer securing member is permeable to water vapour and gasses.

In another embodiment, the ostomy appliance further comprises a water transport layer provided between the inner sealing member and the outer securing member.

In another embodiment, the ostomy appliance further comprises a chafing protection layer provided between the inner sealing member and the outer securing member.

In another embodiment, the ostomy appliance further comprises a water transport layer and a chafing protection layer provided between the inner sealing member and the outer securing member.

In another embodiment, the water transport layer and the chafing protection layer are a single layer.

In another embodiment, the outer securing member comprises urethane tape.

In accordance with another aspect of the present invention, there is provided an ostomy appliance comprising an inner face having an opening which is adapted for receiving a stoma, an outer perimeter, an opposed outer face, an inner sealing member positioned adjacent the opening and water transport layer.

In one embodiment, the layer comprises a woven or non-woven material.

In another embodiment, the layer is adapted for water transport therethrough.

In another embodiment, the ostomy appliance further comprises a chafing protection layer.

In another embodiment, the ostomy appliance further comprises an outer securing member.

In another embodiment, the layer comprises natural fibers or a blend of natural and synthetic fibers.

In accordance with another aspect of the present invention, there is provided an ostomy appliance comprising an inner face having an opening which is adapted for receiving a stoma, an outer perimeter, an opposed outer face, an inner sealing member positioned adjacent the opening and chafing protection layer.

In one embodiment, the layer comprises a woven or non-woven material.

In another embodiment, the ostomy appliance further comprises an outer securing member.

In another embodiment, the layer comprises natural fibers or a blend of natural and synthetic fibers.

### BRIEF DESCRIPTION OF THE DRAWINGS

These and other advantages of the instant invention will be better understood from the following description of the preferred embodiments and the accompanying drawings in which:

FIG. 1 is a perspective view of an individual with a colostomy using an ostomy bag with an outer continuous securing member according to a preferred embodiment the present invention;

FIG. 2 is a perspective view of the assembly of a one piece bag which employs a continuous outer securing member of FIG. 1;

FIG. 3 is an exploded side view of the assembly of FIG. 2;

FIG. 4 is a perspective view of the assembly of a one-piece bag that employs both an outer securing member and a woven or non-woven breathable layer according to another preferred embodiment of the present invention.

FIG. 5 is an exploded side view of the assembly of FIG. 4;

FIG. 6 is a perspective view of an individual with a colostomy wearing an ostomy appliance incorporating the discontinuous securing member according to another aspect of the present invention; and,

FIG. 7 is an exploded side view of the assembly of FIG. 6.

### DETAILED DESCRIPTION OF THE INVENTION

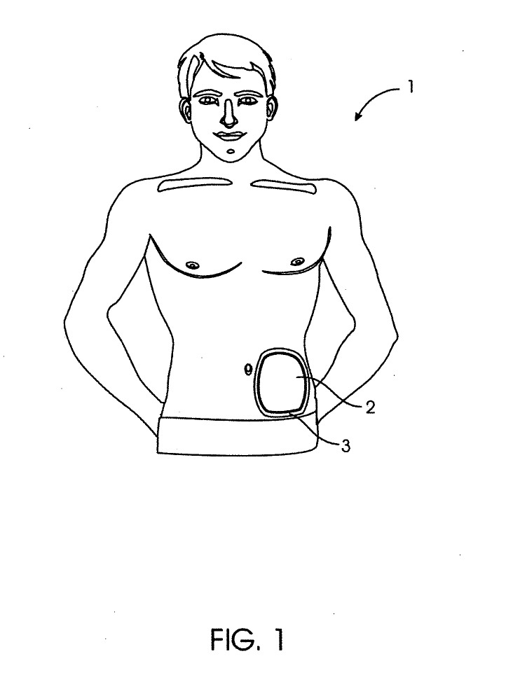

FIG. 1 is a perspective view of an individual 1 with a colostomy using an ostomy bag 2 according to one preferred embodiment that incorporates an outer continuous securing member 3. The outer securing member is located adjacent the outer perimeter of the ostomy bag 2. As continuous securing member 3 secures the outer perimeter of the ostomy bag 2 to the user, a foreign object or a hand cannot be inserted behind the bag. Accordingly the ostomy bag 2 cannot be easily knocked off or be mechanically loosened during work, intimacy, showering or sports including swimming.

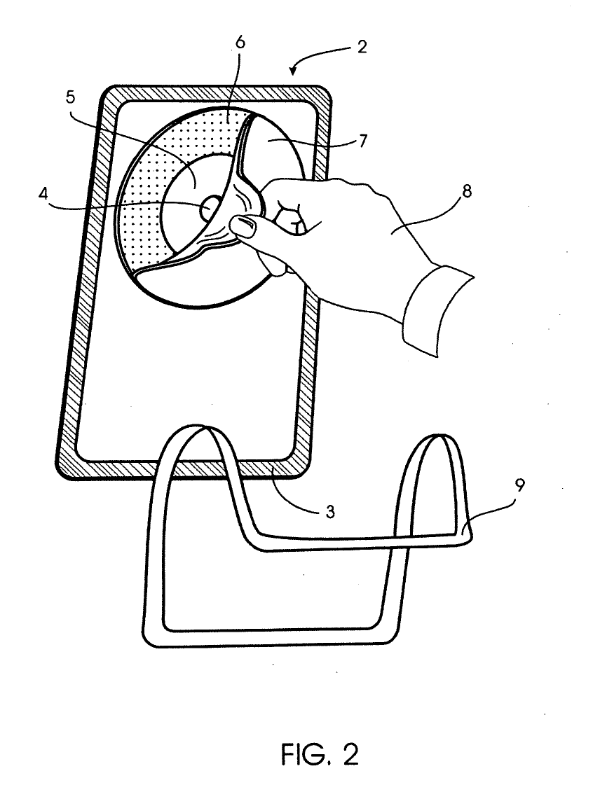

FIG. 2 is a perspective view of the assembly of a one-piece bag 2 that incorporates a hole 4 for the stoma to penetrate, a hydrocolloid tape donut 5, an adhesive tape layer 6, and a securing member 3. As shown in FIG. 2, outer securing member 3 is formed as a continuous annular member that is spaced from adhesive tape layer 6. The adhesive tape layer 6 is protected by release paper 7, which is shown being removed by the hand of the user 8 prior to its application to the body of the user. The outer securing member 3 is protected by release paper 9, which is shown already removed. Alternately, the release papers 7 and 9 can be incorporated into a single element. It will be appreciated that hydrocolloid tape donut 5 and an adhesive tape layer 6 may be any means known in the art to removably secure a stoma in fluid flow communication with ostomy bag 2.

In operation, a user may remove both release papers 7 and 9 and then affix the ostomy bag 2 in place. Alternately, a user may remove release paper 7 and then affix ostomy bag 2 in place. Subsequently, the user may remove release paper 9 and secure the outer perimeter of the ostomy bag 2 in position. The release papers 7, 9 may be removed by the hand 8 of the user after the user has cleaned the stoma and peristomal skin and is ready to attach the bag assembly.

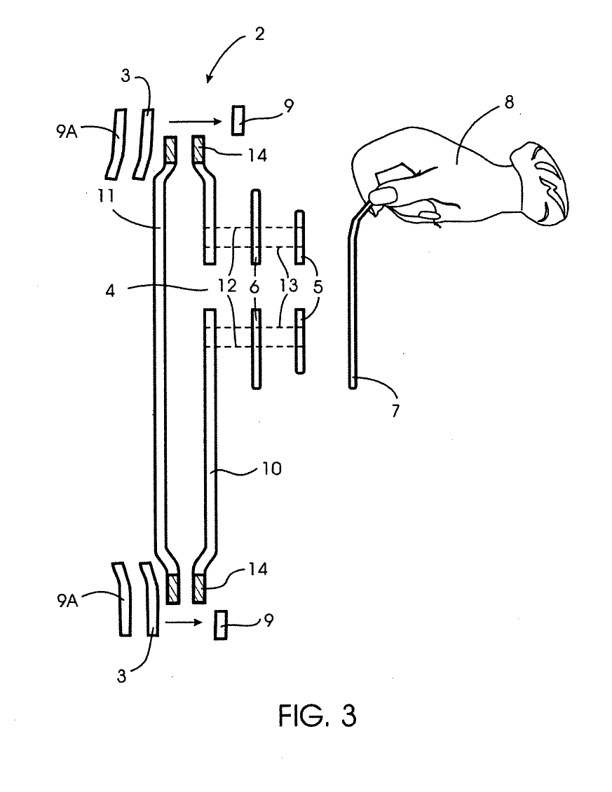

FIG. 3 is a sectional exploded side view of an ostomy bag 2 having a one-piece assembly for connection to a stoma, which utilizes the present invention. The one piece bag 2 is comprised of a subassembly of inner sheet 10 having a hole 4 for the stoma, a hydrocolloid tape donut 5 adhered centrally to a larger adhesive tape ring 6 which may be heat sealed to the thin polyethylene or PVC film or sheet 10 which forms the inner face of the bag 2 while the thin polyethylene or PVC film or sheet 11 forms the opposed outer face of the bag 2. The hydrocolloid tape donut 5 and the larger diameter adhesive tape ring 6 are preferably heat sealed along one or more ridges 12 and 13 to the thin polyethylene or PVC film or sheet 10. Sheet 10 may be heat sealed along the outer perimeter 14 to the thin polyethylene or PVC film or sheet 11 to form the bag 2. The release paper 7 is provided to cover the adhesive side of the hydrocolloid tape donut 5 and the adhesive side of the adhesive tape ring 6 for shipping and handling. Alternately, it will be appreciated that bag 2 may be any construction know in the art.

Outer securing member 3 is preferably made of a urethane material to which a generally waterproof adhesive is applied. The urethane material of which outer continuous securing member 3 is constructed is preferably a thin, flexible adhesive system which stretches without undue pulling of the skin to which it is applied, high permeability to water vapor and oxygen that ensures that the appliance will not fall off after a time, does not leak, does not irritate or damage the skin, does not require frequent changes, and is comfortable to wear for all forms of activities including sports and exercise. The preferred thickness of the urethane adhesive layer is 0.002 to 0.018 inches, more preferable 0.006 to 0.014 inches and most preferably 0.008 to 0.012 inches.

As shown in FIG. 3, outer securing member is an adhesive tape that is applied to overlie outer perimeter 14 of the bag 2. A release paper 9 is provided to cover the adhesive side of outer securing member 3 for shipping and handling. Thus, the outer securing member extends from a position adjacent the perimeter 14 of the opposed outer sheet 11 of the bag 2 outwardly so as to be secured to the skin of the user. Accordingly, when release paper 9 is removed, outer securing member 3 may be secured to the skin of a user. It will be appreciated that outer securing member 3 may extend outwardly from any position on outer sheet 11 and may alternately overlie all of outer sheet 11.

It will be appreciated that, in another embodiment, outer securing member may be provided on inner face 10 outwards of adhesive tape ring 6 and, more preferably, adjacent outer perimeter 14. In such embodiments, outer securing member 3 will provide additional support to secure bag 2 to a user 1. In such a case, outer securing member could be double-sided tape, one side of which is secured to inner sheet 10 and the other side, which faces the user, has an adhesive to be removably affixed to the skin of the user. Alternately, outer securing member could be a liquid adhesive that is applied by the user to inner sheet 10.

In the embodiment that employs a thin flexible tape such as urethane as the outer securing member 3, a ridged support member 9A, that is preferably removable, may optionally be provided to assist in the placement of outer securing member 3. Support member 9A, e.g. paper, assists in keeping sealing member 3 planar while it is applied to the skin of a user so that the sealing member does not fold over itself.

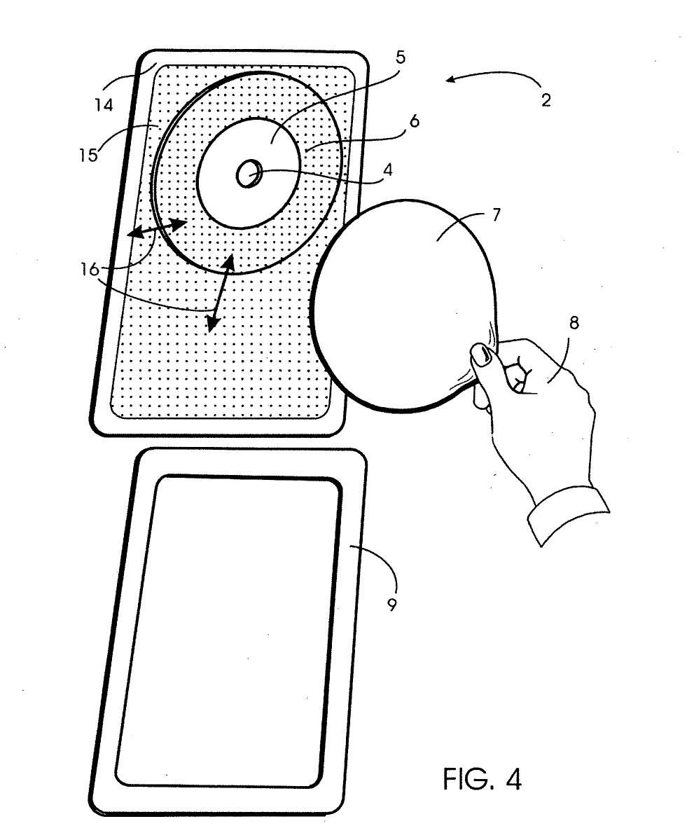

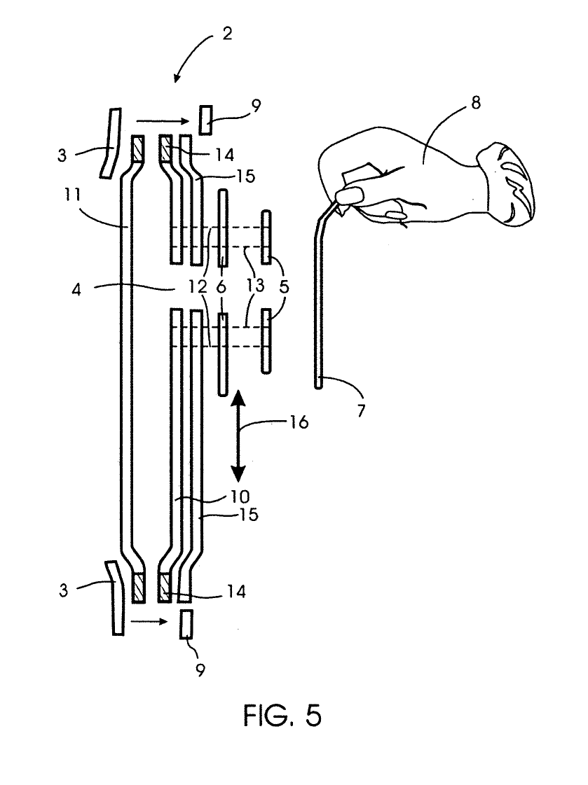

FIGS. 4 and 5 shows an alternate assembly that includes a combiner water transport and chafing protection layer 15. As shown therein, the assembly of a one piece bag 2 incorporates a hole 4 for the stoma to penetrate, a hydrocolloid tape donut 5, an adhesive tape layer 6, a combined water transport and chafing protection layer 15, which may be a woven or non woven material, to allow transverse flow of air and moisture (i.e., generally parallel to the skin of the user as represented by arrow 16) and an outer bag perimeter sealing member 3. The adhesive tape layer 6 is protected by release paper 7, which is shown being removed by the hand of the user 8 prior to its application to the body of the user. The outer securing member 3 is protected by release paper 9 shown already removed. Alternately, the release papers 7 and 9 can be incorporated into a single element. The woven or non-woven layer 15 allows transverse flow of air and moisture (indicated by arrows 16) through the breathable outer securing member 3. A heat seal 14 preferably joins the two layers of the thin polyethylene or PVC film that form the ostomy bag 2.

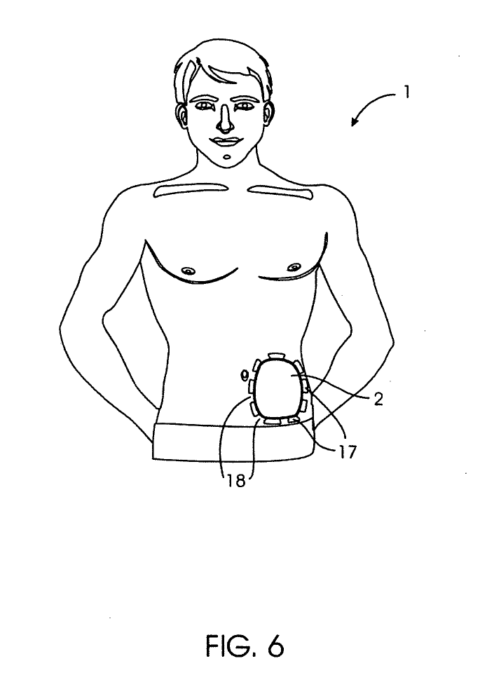

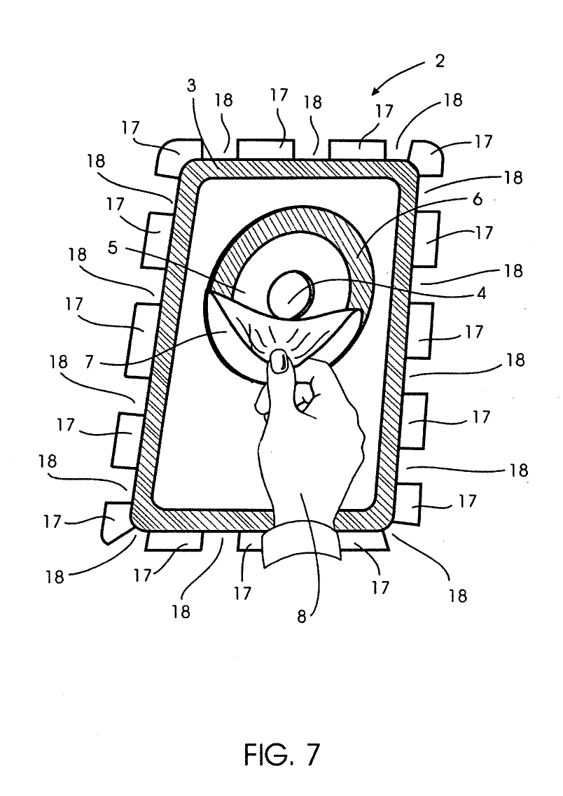

FIGS. 6 and 7 show an individual 1 with a colostomy using an ostomy bag 2 of a further alternate preferred embodiment that incorporates an intermittent or discontinuous outer securing member 17. As the outer securing member is discontinuous, the outer securing member need not be permeable to gasses. Outer securing member 17 may be made of paper, cloth or urethane material to which a waterproof adhesive is applied. The intermittent outer securing member 17 provides openings 18 through which water vapor and oxygen can be transported. It will be appreciated that openings 18 may be sufficiently small so as to prevent a finger or the like entering behind bar 2. As shown in these figures, sealing member 17 comprises a series of strongly adhesive tape segments that encircle the perimeter of the ostomy bag 2. It will be appreciated that the inner ends of the tape segments may be attached to an annular band and extend outwardly therefrom. It will also be appreciated that the tape segments may be formed by selectively removing material from an annular band such as outer securing member 3. It will also be appreciated that openings 18 may be formed by providing small holes through a solid annular band, such as outer securing member 3, or that the tape segments may be independently applied to outer sheet 11 or inner sheet 10.

While the drawings show the invention as it relates to a one-piece colostomy appliance, it is understood that it applies equally to two-piece colostomy appliances, urostomy appliances and ileostomy appliances. While the drawings show the invention as it relates to filter-less bag, it is understood that it applies equally to bags with filters.

1. An ostomy appliance comprising an inner face having an opening that is adapted for receiving a stoma, an outer perimeter, an opposed outer face, an inner sealing member positioned adjacent the opening and an outer securing member spaced from the inner sealing member.

2. The ostomy appliance as claimed in claim 1 wherein the outer securing member is positioned adjacent the outer perimeter.

3. The ostomy appliance as claimed in claim 1 wherein the outer securing member is positioned on the outer face.

4. The ostomy appliance as claimed in claim 1 wherein the outer securing member extends from a position on the outer face that is inwards of the outer perimeter and extends outwardly to a position beyond the outer perimeter.

5. The ostomy appliance as claimed in claim 1 wherein the outer securing member is provided at discrete locations.

6. The ostomy appliance as claimed in claim 1 wherein the outer securing member extends in a continuous annular band.

7. The ostomy appliance as claimed in claim 6 wherein the outer securing member has openings provided therein.

8. The ostomy appliance as claimed in claim 1 wherein the outer securing member is impermeable to liquid water.

9. The ostomy appliance as claimed in claim 8 wherein the outer securing member is permeable to water vapour and gasses.

10. The ostomy appliance as claimed in claim 1 further comprising a water transport layer provided between the inner sealing member and the outer securing member.

11. The ostomy appliance as claimed in claim 1 further comprising a chafing protection layer provided between the inner sealing member and the outer securing member.

12. The ostomy appliance as claimed in claim 1 further comprising a water transport layer and a chafing protection layer provided between the inner sealing member and the outer securing member.

13. The ostomy appliance as claimed in claim 12 wherein the water transport layer and the chafing protection layer are a single layer.

14. The ostomy appliance as claimed in claim 1 wherein the outer securing member comprises urethane tape.

15. An ostomy appliance comprising an inner face having an opening that is adapted for receiving a stoma, an outer perimeter, an opposed outer face, an inner sealing member positioned adjacent the opening and water transport layer.

16. The ostomy appliance as claimed in claim 15 wherein the layer comprises a woven or non-woven material.

17. The ostomy appliance as claimed in claim 15 wherein the layer is adapted for water transport therethrough.

18. The ostomy appliance as claimed in claim 15 further comprising a chafing protection layer.

19. The ostomy appliance as claimed in claim 18 further comprising an outer securing member.

20. The ostomy appliance as claimed in claim 15 further comprising an outer securing member.

21. The ostomy appliance as claimed in claim 20 wherein the outer securing member extends from a position on the outer face that is inwards of the outer perimeter and extends outwardly to a position beyond the outer perimeter.

22. An ostomy appliance comprising an inner face having an opening that is adapted for receiving a stoma, an outer perimeter, an opposed outer face, an inner sealing member positioned adjacent the opening and chafing protection layer.

23. The ostomy appliance as claimed in claim 22 wherein the layer comprises a woven or non-woven material.

24. The ostomy appliance as claimed in claim 23 further comprising an outer securing member.

25. The ostomy appliance as claimed in claim 22 further comprising an outer securing member.

26. The ostomy appliance as claimed in claim 25 wherein the outer securing member extends from a position on the outer face that is inwards of the outer perimeter and extends outwardly to a position beyond the outer perimeter.
